(19) United States
(12) Patent Application Publication
(10) Pub. No.: US 2007/0073263 A1
(43) Pub. Date: Mar. 29, 2007

(54) Infusion bags having a medicine mixing nozzle with a puncture function

(76) Inventors: Xianghua Liu, Changsha City (CN); Xunde Peng, Changsha City (CN)

Correspondence Address: HANLEY, FLIGHT & ZIMMERMAN, LLC, 150 S. WACKER DRIVE, SUITE 2100, CHICAGO, IL, 60606, US

(21) Appl. No.: 11/521,687

(22) Filed: Sep. 15, 2006

Related U.S. Application Data
(63) Continuation of application No. PCT/CN04/00530, filed on May 24, 2004.

(30) Foreign Application Priority Data
Mar. 18, 2004 (CN) 200410022990.5

(51) Int. Cl. A61B 19/00

(52) U.S. Cl. 604/408; 604/403

(57) ABSTRACT

Infusion bags including a mixing medicine nozzle with puncture functionality and methods of using the same are disclosed. An example infusion bag comprises: a bag body having a medicine containing chamber; a medicine mixing tube having a first end in communication with the medicine containing chamber of the bag body; and a medicine output nozzle in communication with the medicine containing chamber of the bag body via a medicine output tube. A second end of the medicine mixing tube is connected to a medicine mixing nozzle, a bottom of the medicine mixing nozzle is covered with a protection membrane, and a needle tube in communication with the medicine mixing tube is disposed in an inner chamber of the medicine mixing nozzle.

### RELATED APPLICATION

This Patent arises from a continuation of International Patent Application Ser. No. PCT/CN2004/000530, which was filed on May 24, 2004, and which is hereby incorporated by reference in its entirety.

### FIELD OF THE DISCLOSURE

The present disclosure relates generally to the field of medicine packaging, and more particularly to medicine bags for infusion.

### BACKGROUND

Medicine mixing methods in current clinical use include the following three modes. In a first mode, a medicine output nozzle and a medicine mixing nozzle are provided on a bag. At the time of mixing the medicine, an injector is used to draw out the medicinal liquid from the bag, and then to inject the medicinal liquid into a cillin bottle containing powdery or aqueous medicinal preparations. The above procedure is repeated until there is enough medicinal liquid in the cillin bottle. The bottle is subsequently repetitively shaken until the medicine in the bottle is evenly mixed. Finally, the medicine in the bottle is drawn out by the injector and then injected into an infusion bag. This procedure repeats several times until the medicine in the bottle is entirely drawn out. This mode of medicine mixing is currently used in most clinical applications.

In a second mode, an independent medicine mixing nozzle is used, which consists of a connection sheath and a needle tube. When in use, the needle tube of the medicine mixing nozzle is first inserted into the cillin bottle, and the other end of the needle tube is inserted into the medicine mixing nozzle on the infusion bag. Because the needle tube is not very tightly connected to the medicine mixing nozzle on the infusion tube, and also because the inner aperture of the needle tube is very small, the medicinal liquid tends to leak along the outer wall of the needle tube due to heavy pressure when the infusion bag is pressed against, thereby contaminating the operating hand and the infusion bag and hence encumbering use of the bag. Therefore, this method is seldom used.

In a third mode, an infusion bag having a medicine mixing nozzle is used, which infusion bag mainly comprises a bag body, a medicine output nozzle and a medicine mixing nozzle. Since its medicine mixing nozzle is configured to be in an open state without sealing, if the medicine mixing nozzle is contaminated before use, (especially contaminations in comer areas of the needle tube), disinfection is impossible, thereby rendering it impossible to widely use such a method.

### BRIEF DESCRIPTION OF THE DRAWINGS

FIG. 1 is a structural illustration of an example infusion bag constructed in accordance with the teachings of the invention.

FIG. 2 illustrates the structure of a first example medicine mixing nozzle constructed according to the teachings of the invention.

FIG. 3 illustrates the structure of a second example medicine mixing nozzle constructed according to the teachings of the invention.

FIG. 4 illustrates the structure of a third example medicine mixing nozzle constructed according to the teachings of invention.

In the drawings, the following reference numerals refer to the following structures:

1. bag body

2. medicine output nozzle

3. medicine mixing nozzle

4. clamp

5. medicine mixing tube

6. needle tube

7. medicine output tube

8. protection membrane

9. connection sheath

10. sealing nozzle

11. inner plug

### DETAILED DESCRIPTION

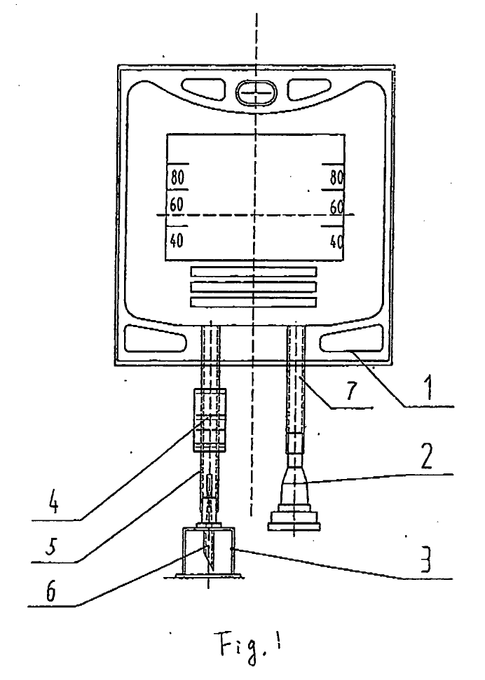

As shown in FIG. 1, an example infusion bag comprises a bag body 1, a medicine output nozzle 2, a medicine mixing tube 5 and a medicine output tube 7. A first end of the medicine mixing tube 5 communicates with the medicine containing chamber of the bag body 1. A first end of the medicine output nozzle 2 also communicates with the medicine containing chamber of the bag body 1 via the medicine output tube 7. A second end of the medicine output nozzle 2 is sealed. A second end of the medicine mixing tube 5 is connected to a medicine mixing nozzle 3, whose lower end is configured to be a cylindrical structure with the periphery sealed to facilitate insertion of a cillin bottle nozzle and also to avoid contamination. A needle tube 6 is disposed inside the inner chamber of the cylindrical body of the medicine mixing nozzle 3. The needle tube 6 is communicable with the medicinal liquid chamber via the medicine mixing tube 5. The needle tube 6 can puncture into the cillin bottle in order to draw medicinal liquid when in use to subsequently put the latter into the medicine chamber. The lower end face of the medicine mixing nozzle 3 is covered with a protection membrane 8, which seals the inner chamber of the medicine mixing nozzle 3 to prevent the needle tube 6 from being contaminated during transportation and packaging.

To prevent medicinal liquid from being spilt out of the medicine mixing nozzle 3 during infusion, a clamp 4 is disposed on the medicine mixing tube 5 to affect opening and closing of the passage of the medicine mixing tube 5.

The medicine mixing nozzle 3 can be configured as shown in FIG. 2. In the example of FIG. 2, the medicine mixing nozzle 3 comprises a protection membrane 8, a connection sheath 9, a sealing nozzle 10 and a needle tube 6. The protection membrane 8 covers the bottom of the connection sheath 9. The sealing nozzle 10 is fixed on an upper end of the connection sheath 9. The needle tube 6 is disposed inside an inner chamber at a lower end of the connection sheath 9. The needle tube 6 is connected to the sealing nozzle 10 with its upper end. The juncture between the needle tube 6 and the sealing nozzle 10 is processed with creases to releasably block communication between the tube 6 and the nozzle 10. When in use the juncture connecting the sealing nozzle 10 and the needle tube 6 is first broken by folding so that the needle tube 6 is in communication with its two ends, thereby making it possible for the medicinal liquid in the inner chamber of the cylindrical body of the medicine mixing nozzle 3 to be entirely injected into the infusion bag. The connection sheath 9, the sealing nozzle 10 and the needle tube 6 can be integrally formed by one injection molding, so as to avoid leakage at the positions of the interfaces and contamination during assemblage.

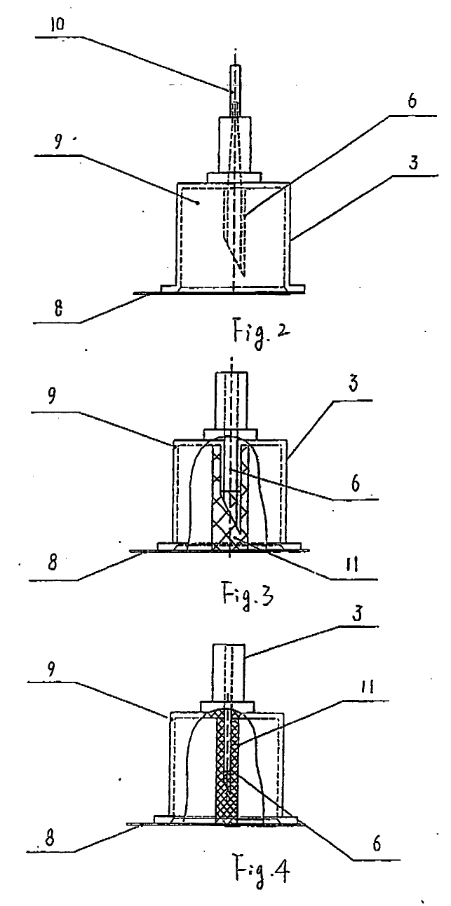

As shown in FIG. 3, the medicine mixing nozzle 3 can alternatively comprise a protection membrane 8, a connection sheath 9, an inner plug 11 and a needle tube 6. In the example of FIG. 3, the needle tube 6 is placed inside an inner chamber at a lower end of the connection sheath 9. The inner plug 11 sheathes the needle tube 6 to protect the latter and prevent it from being contaminated. The protection membrane 8 likewise covers the bottom of the connection sheath 9, thus avoiding contamination of the entire inner chamber of the connection sheath 9. The connection sheath 9 and the needle tube 6 can be constructed as an integral piece by means of, for example, injection molding. When in use, it suffices to first tear out the protection membrane 8 and then pull out the inner plug 11. In comparison with the medicine mixing nozzle 3 shown in FIG. 2, the medicine mixing nozzle 3 shown in FIG. 3 is more convenient and reliable in use.

The medicine mixing nozzle 3 can alternatively be configured as shown in FIG. 4. In the example of FIG. 4, the nozzle 3 comprises a protection membrane 8, a connection sheath 9, an inner plug 11 and a needle tube 6. The needle tube 6 may be made of a stainless steel material and may be integrally formed with the connection sheath 9 via injection molding. To employ the nozzle 3 of FIG. 4, one pulls out the inner plug 11.

In use: in medicine mixing, the protection membrane 8 on the medicine mixing nozzle 3 is first removed. The needle tube 6 on the medicine mixing nozzle 3 then punctures the rubber plug of the cillin bottle to place the infusion bag in communication with the cillin bottle. Subsequently, the infusion bag is placed higher than the cillin bottle and is repetitively pressed against by hand until the medicinal liquid in the cillin bottle is sufficient to evenly mix the medicine in the bottle. Then the cillin bottle is placed higher than the infusion bag to repeat the aforementioned action until all medicinal liquid in the bottle is drawn back into the infusion bag. Finally, the clamp 4 is used to clamp tightly against the passage of the medicine mixing tube 5 to close it, thus finishing the medicine mixing procedure.

From the foregoing, persons of ordinary skill in the art will appreciate that the above disclosed methods and apparatus overcome the aforementioned defects in the prior art, by providing an infusion bag that is directly communicative with a cillin bottle without any contamination by means of a medicine mixing nozzle having a puncture function.

A disclosed example infusion bag comprises a bag body, a medicine mixing tube with a first end in communication with a medicine containing chamber of the bag body, and a medicine output nozzle also in communication with the medicine containing chamber of the bag body via a medicine output tube. A second end of the medicine mixing tube is connected to the medicine mixing nozzle, and, at the bottom of the medicine mixing nozzle, it is covered with a protection membrane. A needle tube is disposed in an inner chamber of the medicine mixing nozzle. The needle tube is communicable with the medicine mixing tube. A clamp is disposed on the medicine mixing tube for opening and closing the tube. The medicine mixing nozzle can comprise a protection membrane, a connection sheath, a sealing nozzle and a needle tube. The protection membrane covers the bottom of the connection sheath, the sealing nozzle is disposed at an upper end of the connection sheath, and the needle tube is disposed inside an inner chamber of the connection sheath and connected to the sealing nozzle. The juncture between the connection sheath and the sealing nozzle is processed with creases which can be broken to open communication into the bag body.

The medicine mixing nozzle can alternatively comprise a protection membrane, a connection sheath, an inner plug and a needle tube. In such an approach, the needle tube is placed inside an inner chamber of the connection sheath, the inner plug sheathes the needle tube, and the protection membrane covers the bottom of the connection sheath.

This disclosed example methods and apparatus have the following technical effects: (1) as a medicine mixing nozzle with puncture function is provided on the infusion bag, a disposable injector and cotton swabs for sanitizing are not needed in the medicine mixing procedure. This saves cost, reduces secondary pollution of medicinal liquid, simultaneously simplifies the medicine mixing procedure, and makes the medicine mixing operation convenient and easy; (2) the medicine mixing nozzle and the infusion bag are formed integrally, so the leak phenomenon when an independent medicine mixing nozzle and an infusion bag are used simultaneously is avoided; and (3) after the medicinal liquid is prepared, the cillin bottle can still hang on the infusion bag, and the security and credibility of medicine distribution may be enhanced.

Although certain example methods, apparatus and articles of manufacture have been described herein, the scope of coverage of this patent is not limited thereto. On the contrary, this patent covers all methods, apparatus and articles of manufacture fairly falling within the scope of the appended claims either literally or under the doctrine of equivalents.

What is claimed is:

1. An infusion bag having a medicine mixing nozzle with puncture function, comprising:
a bag body having a medicine containing chamber;
a medicine mixing tube having a first end in communication with the medicine containing chamber of the bag body; and
a medicine output nozzle in communication with the medicine containing chamber of the bag body via a medicine output tube, wherein a second end of the medicine mixing tube is connected to a medicine mixing nozzle, a bottom of the medicine mixing nozzle is covered with a protection membrane, and a needle tube in communication with the medicine mixing tube is disposed in an inner chamber of the medicine mixing nozzle.

2. The infusion bag as defined in claim 1, wherein a clamp is disposed on the medicine mixing tube.

3. The infusion bag as defined in claim 1, wherein the medicine mixing nozzle comprises a protection membrane, a connection sheath, a sealing nozzle, and a needle tube, wherein the protection membrane covers a bottom of the connection sheath, the sealing nozzle is located at an upper end of the connection sheath, and the needle tube is disposed inside an inner chamber of the connection sheath and connected to the sealing nozzle, and a juncture between the connection sheath and the sealing nozzle is creased.

4. The infusion bag as defined in claim 1, wherein the medicine mixing nozzle comprises a protection membrane, a connection sheath, an inner plug and a needle tube, wherein the needle tube is disposed inside an inner chamber of the connection sheath, the inner plug sheathes the needle tube, and the protection membrane covers a bottom of the connection sheath.
